(19) United States
(12) Patent Application Publication
(10) Pub. No.: US 2015/0017203 A1
(43) Pub. Date: Jan. 15, 2015

(54) COMBINATION PRODUCT FOR TREATING EXCESS WEIGHT AND/OR FOR IMPROVING THE FIGURE

(71) Applicant: INSTITUT NATIONAL DE LA RECHERCHE AGRONOMIQUE - INRA, PARIS (FR)

(72) Inventors: Beatrice Morio-Liondore, Saint George Sur Allier (FR); Frederic Capel, Romagnat (FR); Jean-Pascal De Bandt, Croissy Sur Seine (FR); Christophe Moinard, Bourg La Reine (FR); Luc Cynober, Sceaux (FR)

(73) Assignees: UNIVERSITE PARIS DESCARTES, Paris (FR); ASSISTANCE PUBLIQUE HOPITAUX DE PARIS, Paris (FR); INSTITUT NATIONAL DE LA RECHERCHE AGRONOMIQUE - INRA, Paris (FR)

(21) Appl. No.: 14/381,760

(22) PCT Filed: Feb. 28, 2013
(86) PCT No.: PCT/FR2013/050430

§ 371 Date: Aug. 28, 2014

(30) Foreign Application Priority Data
Feb. 29, 2012 (FR) 1251832

(51) Int. Cl. A61K 31/40; A61K 45/06; A61K 31/198

(52) U.S. Cl. CPC A61K 31/40; A61K 31/198; A61K 45/06
USPC 424/195.15; 514/423

(57) ABSTRACT

A combination product includes as active substances, at least an inhibitor of the HMG-CoA-reductase enzyme and citrulline, or a bioequivalent compound thereof, for treating excess weight or obesity and/or the accumulation of body fat. A cosmetic treatment method for improving or slimming the figure and/or for stimulating the loss of excess weight and/or of cellulite and/or for limiting the accumulation thereof, includes the administration of a combination product including as active substances, at least an inhibitor of the HMG-CoA-reductase enzyme or a physiologically acceptable salt thereof, and citrulline, or a bioequivalent compound thereof, or one of theirs analogues, and the renewal of the administration until the expected cosmetic effect is obtained. The combination product such as previously defined for improving or slimming the figure and/or for stimulating the loss of excess weight and/or of cellulite and/or for limiting the accumulation thereof is also described.

### FIELD OF THE INVENTION

The present invention relates to the field of products dedicated to the treatment of excess body fat and/or of excess weight or obesity. The present invention also relates to cosmetic treatment methods for reducing or preventing the accumulation of body fat and for improving the figure.

### PRIOR ART

Defined as a medical condition in which excess body fat has accumulated to the extent that it may have a negative effect on health, obesity is one of the main priorities of the World Health Organization (WHO), as regards disease prevalence and management. Indeed, on a world scale, 300 million people are obese, thus representing about 15 to 30% of the population in industrialized countries. It is also estimated that 43 million kids under the age of five are overweight and thus have a higher risk of developing obesity once they become adults. Moreover the increasing prevalence of obesity in the global population goes along with an increased proportion of excess weight mothers or obese women at the start of their pregnancies. Yet obese mothers are much more likely to give birth to obese children, especially if they suffer from gestational diabetes or from metabolic syndrome of pregnancy. In France, excess weight and obesity together affect more than 46% of adults and 19% of children.

Excess weight (or overweight) and obesity are commonly defined using a body mass index (BMI), based on the weight and the height of an individual to categorize the corpulence thereof. Expressed in kg/m², the BMI corresponds to the individual's body mass divided by the square of the height thereof. According to the WHO standards, for a human adult, a BMI ranging from 18.5 to 25 kg/m² corresponds to a corpulence that is said to be <<normal>>, that is to say that is not associated with an increase in burden of disease (negative context effects on health). Excess weight is characterized through a BMI from 25 to 30 kg/m², and obesity through a BMI over 30 kg/m².

Adipose tissue (or body fat) is a connective tissue comprising fat cells, or adipocytes, connected through a thin fibrous tissue. Immediately below the skin, adipose tissue does protect and isolate most part of the human body, while also acting as an energy reserve. The body fat develops by multiplying the number of the fat cells (adipocyte hyperplasia) and/or by increasing the cell size (adipocyte hypertrophy). In the adult, the adipose tissue mass mainly increases through the adipocyte hyperplasia mechanism, more rarely through hyperplasia. The body fat percentage may be estimated using various methods known from the person skilled in the art. One considers that for men, a body fat percentage from 15 to 20% is within the range for normality, that is to say is not associated with a pathological condition. For women, normal rates do range from 25 to 30%. Beyond these respective figures for men and women, excess body fat may be associated with severe pathological conditions. The impact of excess body fat (adipose tissue) on health also depends on the body location thereof. Indeed, body fat distribution does vary in many ways. Thus a gynoid type fat distribution is defined as a fat mass accumulation at the hip, buttock and leg levels and an android type fat distribution as a fat mass accumulation at the abdominal organs level, on the abdominal wall and sometimes on the back upper part. Epidemiologic studies show that the risk of developing metabolic diseases and/or cardiovascular diseases is especially linked to an android type excess weight and obesity (abdominal adiposity). In this respect, abdominal obesity (or android obesity) is the central factor for defining the metabolic syndrome and as such is associated with a strong morbidity. Excess weight and obesity are therefore also diagnosed using the waist circumference measurement, or the waist circumference/hip circumference ratio, so as to directly estimate the abdominal adiposity and the occurrence of an android type excess weight or obesity. For women, excess weight is characterized through a waist circumference ranging from 80 to 88 cm, and obesity through a waist circumference over 88 cm. For men, excess weight is characterized through a waist circumference ranging from 94 to 102 cm, obesity through a waist circumference over 102 cm. Considering the waist circumference/hip circumference ratio, obesity is defined according to the WHO standards for men through a ratio over 0.9 and for women through a ratio over 0.85. For children and teenagers, the BMI also enables to evaluate the corpulence. During childhood, the BMI does physiologically vary with age and sex. BMI values should thus be reported onto percentile curves illustrating the corpulence evolution. In 2000, the International Obesity Task Force (IOTF), under the aegis of the WHO, proposed a world-wide definition of childhood obesity (Cole and al., BMJ., 2000, 320(7244):1240-3). Excess weight thresholds (class I obesity) and obesity thresholds (class II obesity) for children are respectively defined through percentile curves meeting respectively the BMI of 25 and the BMI of 30 at the age of 18. These curves enable to show the child-adult continuity. The National Cholesterol Education Program Adult Treatment Panel III (NCEP-ATP III) also retains as a criterion for obesity in children or teenagers a waist circumference over 90 cm.

In Europe, two main risk factors especially contribute to impair the energy balance and to produce excess weight or even obesity or to increase the fat mass: (i) a high-energy density diet delivering a low satiety feeling, resulting from a high proportion of energy originating from fats (vegetable oils and animal fats) (i.e., hyperlipidic diet), from a high sugar consumption and a poor dietary fibers absorption, together with an increased consumption of high-sugar content beverages, optionally with an alcohol fraction and (ii) a low energy expenditure linked to a more sedentary lifestyle. Both factors are no longer under the control of the normal and quick-acting biological regulation of appetite and food absorption, so that the individuals tend not to adapt their consumption when those foods and drinks are proposed all the time.

Various other risk factors may also be mentioned, such as anxiety, stress or depression, a familial susceptibility to weight gain or to obesity (involving genetic markers), hormonal imbalance or menopause.

Adapted dietetic and hygienic measures, combining a balanced diet and a regular physical activity, do represent the simplest way to fight against excess weight or obesity. Yet practicing a regular physical activity is constraining and, moreover, the benefits of a restrictive diet are often limited by a weight rebound effect when starting normally eating and drinking again.

Therefore, it is important to develop complementary treatments to prevent and treat weight severe disorders, and especially abdominal fat accumulation, the impact of which is highly worrying in terms of morbidity and mortality.

Moreover, the weight loss, mainly induced through restrictive diets, is associated with a substantial muscle loss (lean body mass). This muscular atrophy for obvious medical reasons must of course be prevented because it is always an indication for a poor prognosis.

Localized fat accumulations (i.e. body fat or adipose tissue), as for example, cellulite, may also participate to the condition in a non-pathological manner (i.e.: non associated with an increased burden of disease) in individuals being in good health with a normal corpulence (according to the WHO standards). Such fatty accumulations, although not directly affecting the health, may be considered as being unaesthetic.

For women, for example, body fat accumulates especially at the buttock and hips levels. Such an accumulation becomes even stronger during menopause.

Therefore it is also useful to develop cosmetic strategies to allow people who are in good health to stabilize a weight as low as possible, as long as these values do not have a pathological character, and/or to stay thin with a figure having no localized fat deposits, such as cellulite, or also to loss excess weight and/or an ungraceful body fat, like cellulite. Such a cosmetic treatment could also be aimed at women going through menopause, to prevent and/or restrain the gynoid or android type body fat accumulation, as well as at young women who want to eliminate unaesthetic extra deposits of body fat, for example post pregnancy.

Such therapeutic or cosmetic strategies will be all the more useful as they will make it possible to preferably target a body fat loss while preserving the lean body mass.

### SUMMARY OF THE INVENTION

The present invention relates to a combination product, including as active substances, at least:

(i) an inhibitor of the HMG-CoA-reductase enzyme, and

(ii) citrulline;

for use in treating excess weight or obesity and/or the accumulation of body fat.
In a particular embodiment of the present invention, the presence of arginine in a combination product of the invention is excluded.
In an embodiment of the invention, said combination product is characterized in that the active substances consist in:
i. an inhibitor of the HMG-CoA-reductase enzyme, and
ii. citrulline.
In some embodiments, the combination product of the invention includes, as active substances, at least:
(i) atorvastatin or a statin of natural origin, or a plant extract containing the same, as for example a red yeast rice extract, and
(ii) citrulline.

In some embodiments, the combination product of the present invention is characterized in that citrulline is administered at a daily dosage of from 1 to 15 g/day.

In some embodiments of the present invention, the combination product of the invention may come as a single (or combined) composition including as active substances at least:

(i) an inhibitor of the HMG-CoA-reductase enzyme, and

(ii) citrulline;

in a physiologically acceptable medium.

In particular, the combination product of the invention may come as a single composition the active substances (i) and (ii) of which consist in:

(i) an inhibitor of the HMG-CoA-reductase enzyme, and

(ii) citrulline and/or a bioequivalent compound thereof;

in a physiologically acceptable medium.

In other embodiments, the combination product of the invention may come as a kit including:

(a) a composition including at least an inhibitor of the HMG-CoA-reductase enzyme in a physiologically acceptable medium; and
(b) a composition including at least citrulline, in a physiologically acceptable medium;
said compositions (a) and (b) being each provided in a form adapted to a sequential, a separate and/or a simultaneous administration with one another over time.
In a particular embodiment, the combination product of the invention may come as a kit including:
(a) a composition consisting in an inhibitor of the HMG-CoA-reductase enzyme in a physiologically acceptable medium; and
(b) a composition consisting in citrulline, in a physiologically acceptable medium;
said compositions (a) and (b) being each provided in a form adapted to a sequential, a separate and/or a simultaneous administration with one another over time.

In a particular embodiment of the present invention, the combination product is characterized in that the active substances (i) and (ii) or the compositions (a) and (b) are in a medium or in media which are adapted to oral administration.

When used in the therapeutic treatment of obesity or excess weight and body fat accumulation, the combination product according to the present invention especially aims at people who are stressed or anxious, and/or having a diet with a high fat or a high sugar content, and/or a sedentary lifestyle, and/or suffering from hormonal imbalances and/or from a genetic susceptibility to excess weight and/or obesity, or taking prescription drugs which can cause weight gain.

In some particularly preferred embodiments, the combination product of the invention is aimed at patients suffering from glucose metabolism disorders, and especially from a high fasting and/or postprandial hyperglycemia, considering that the fasting and/or postprandial glycemia levels are lower than those defining a diabetic condition.

Citrulline can be present as the active substance (ii) in a combination product of the present invention in a more or less purified form, especially in the form of a plant extract. It can also come in a free form or as derivatives. Citrulline derivatives that are acceptable according to the present invention especially include complex forms, protected forms (like esterified forms and forms wherein the amine function is protected by the tert-butoxycarbonyl group, i.e. Boc) or salts, as well as precursors thereof, like ornithine or glutamine. As an example, a combination product of the invention may include, as an active substance (ii), at least one substance selected from the group consisting of: D,L-citrulline, L-citrulline, D,L-citrulline malate, L-citrulline malate, L-citrulline monoacetate, L-citrulline hydrochloride, L-citrulline methylester, L-citrulline ethylester, L-citrulline-n-hexylester, L-citrulline (benzoylmethyl)ester, alpha-N-benzoyl-L-citrulline methylester, B-Boc-citrulline, N1-2,4-dinitrophenyl-D,L-citrulline and their mixtures in whatever proportions.

Citrulline may come in a purified form or as a plant extract containing the same, especially as a plant extract belonging to the cucurbitaceae plant family.

Typically, a product of the invention is characterized in that the active substance (i) is administered at a daily dose ranging from 10 to 80 mg/day, when using a synthetic statin and the active substance (ii) is administered at a daily dose ranging from 2 to 5 g/day.

The HMG-CoA reductase inhibitor may especially be selected from the group consisting of natural or synthetic statins, monacolins, berberin, and policosanols and their mixtures in whatever proportions.

A combination product especially adapted to the present invention contains at least atorvastatin as the HMG-CoA reductase inhibitor.

In other advantageous embodiments of the present invention, the HMG-CoA reductase inhibitor is a natural statin, or comes as a rice red yeast extract, or monascus purpureus.

The present invention further relates to a combination product comprising as active substances at least:

(i) a HMG-CoA reductase inhibitor in the form of a plant extract containing the same, preferably an extract of rice red yeast, and/or a plant extract including monacolins and/or berberin and/or policosanols, and

(ii) citrulline preferably in the form of a plant extract containing the same;

said substances (i) and (ii) being in a form adapted to a sequential, a separate and/or a simultaneous administration with each other.
In a particular embodiment of said product, citrulline is in a pure form.
In another embodiment, the combination product comprises active substances consisting in:

(i) a HMG-CoA reductase inhibitor in the form of a plant extract containing the same, preferably a red yeast rice extract, and/or a plant extract including monacolins and/or berberin and/or policosanols, and

(ii) citrulline, in the form of a plant extract containing the same or in a pure form;

said substances (i) and (ii) being in a form adapted to a sequential, a separate and/or a simultaneous oral administration with each other.
Such a product may come as a single composition or as distinct compositions, as previously described.

In an advantageous embodiment, the active substances (i) and (ii) are in a medium or in media, according to whether the combination product is respectively in the form of a single composition or in the form of distinct compositions, adapted to oral administration.

As an example, in such a combination product, composition (a) may come as a capsule dosage unit and composition (b) may come as an individual sachet.

Preferably, such a combination product is characterized in that the active substance (i) is a standardized extract including 2% of monacolin K.

The present invention further relates to a cosmetic treatment method, for improving the appearance of individuals, for example for slimming the figure and/or reducing or restraining localized fat accumulations or lipodystrophy, and/or for stimulating the loss of excess weight and/or of cellulite, and/or for limiting the accumulation thereof, in individuals who are in good health, comprising the administration of a combination comprising as active substances at least:

(i) a HMG-CoA reductase inhibitor in the form of a plant extract containing the same, preferably a red yeast rice extract, and/or a plant extract including monacolins and/or berberin and/or policosanols;

(ii) citrulline, preferably in the form of a plant extract containing the same;

and the renewal of said administration until the expected cosmetic effect is obtained. Preferably, said association comes as a combination product wherein said substances (i) and (ii) are in a form adapted to a sequential, a separate and/or a simultaneous oral administration with one another over time.

Preferably, in such a cosmetic treatment method the active substance (i) is administered at a dose ranging from 100 to 600 mg one to three times a day and the active substance (ii) is administered at a daily dose, ranging from 2 to 5 g/day.

Such a cosmetic treatment is aimed at individuals whose weight and excess fat are not associated with a growing burden of disease and in particular at individuals who do not suffer from glucose metabolism disorders, insulin resistance, any metabolic syndrome, diabetes or vascular disorders.

The present invention finally relates to the use of an association comprising at least (i) a HMG-CoA reductase inhibitor, and

(ii) citrulline,
for improving the appearance of individuals, for example for slimming the figure and/or reducing or restraining localized fat accumulations, or lipodystrophia, and/or for stimulating the loss of excess weight and/or of cellulite and/or for limiting the accumulation thereof, in individuals who are in good health. Preferably, said association will be in the form of a combination product such as previously defined. Still preferably, the combination product will be adapted to oral administration.

The various embodiments of the present invention are especially described in the detailed specification hereafter. These embodiments may be considered separately or be combined with each other.

### DETAILED DESCRIPTION OF THE INVENTION

In the present invention, the applicant evidenced that said association containing at least (i) a HMG-CoA reductase inhibitor and (ii) citrulline, has a synergistic effect on weight loss and body fat reduction, while preserving the lean body mass.

As used herein, a <<synergistic effect>> is intended to mean that said association comprising at least a HMG-CoA reductase inhibitor and citrulline has an impact on weight loss or body fat reduction, during the treatment period which is stronger than the cumulative effects of a treatment (a) comprising the administration of a HMG-CoA reductase inhibitor, non associated with citrulline, and conversely of a treatment (b) comprising the administration of citrulline, non associated with a HMG-CoA reductase inhibitor.

Citrulline is an intermediate produced during the urea cycle in the liver, during which excess ammonia is removed. As a precursor of arginine it would also be involved in nitrogen monoxide synthesis (Curis E. and al., Amino Acids, 2005 Nov., 29(3): 177-205). Some studies do suggest that citrulline could have an effect on the lean body mass. Indeed, citrulline could stimulate protein synthesis and especially would modulate the metabolism of the muscle proteins (Osowska S. and al., Citrulline modulates muscle protein metabolism in old malnourished rats, Am. J. Physiol. Endocrinol. Metab., 2006 April). A recent scientific publication mentioned the nitrogen monoxide regulating role towards the energy metabolism and suggested that arginine or its precursor citrulline could play a role in mammals (animals and/or humans) for a preventive action onto, or for the treatment of, the metabolic syndrome in obeses and could contribute to reduce the body fat proportion in farmed animals (Jobgen W. S. and al., J. Nutr. Biochem., 2006; 17(9): 571-88).

Inhibitors of the HMG-CoA-reductase enzyme, and in particular statins, are lipid-lowering agents that are especially prescribed in the treatment of dyslipemias, in particular hypercholesterolemia, for the primary and secondary prevention of cardiovascular events. However, a possible effect of the inhibitors of the HMG-CoA reductase enzyme on the body fat could not be demonstrated until now, as far as the applicant knows. On the contrary, a study from Bucek and al., (Vasa, J. for Vascular Disease, german, 2006, 35(2):92-5) reported that the results of clinical assays about the influence of inhibitors of the HMG-CoA-reductase enzyme on the body fat concluded that taking statin, in particular atorvastatin and simvastatin, in the long term has not any positive influence on body fat.

CN 101731699 describes compositions comprising many various active substances, like apple juice, carrot, lemon and rice red yeast. Many therapeutic and cosmetic indications are proposed and especially with an anorexigenic purpose, which necessarily would result in a lean body mass loss.

CN 20091265500 also describes compositions especially aiming at producing anorectic effects, comprising various components, like black rice powder, fibers, soybean, bean powder, pumpkin powder and taurine.

US 2005/0288737 suggests to associate arginine with a HMG-CoA reductase inhibitor for treating excess weight, but excludes the association of only citrulline with a HMG-CoA reductase inhibitor.

It is thus substantially surprising to be able to demonstrate according to the present invention that such an association containing at least (i) a HMG-CoA reductase inhibitor and (ii) citrulline has a synergistic effect on weight loss and on body fat reduction without any lean body mass loss. Such an association will thus be dedicated to the (preventive or curative) treatment of excess weight or obesity and/or excess body fat. In particular, a combination product such as defined in the present application and associating (i) a HMG-CoA reductase inhibitor and (ii) citrulline will be used for limiting the body fat gain or reducing the stubborn fat without affecting the lean body mass, in the treatment of excess weight and/or obesity.

Such a combination product could also be used within the framework of a cosmetic treatment process (or method) in people who are in good health, that is to say whose weight and/or excess body fat are not associated with a growing burden of disease. As used herein, <<people in good health>> preferably mean individuals who have a corpulence considered as normal according to the WHO standards. When used with a cosmetic approach, said association of the invention will preferably be proposed in a form that is adapted to oral administration, as for example a food supplement or a functional food.

As used hereafter in the present application, a <<combination product>> is also used as an equivalent to <<said association>> such as defined hereabove.

Citrulline, as used in the present invention, does refer to the D, L enantiomer forms as well as their mixtures in whatever proportions. In a combination product of the invention, citrulline may come in a pure form and/or be contained in a plant extract. It may be in a free form and/or as at least one of the derivatives thereof. Said derivatives consist in citrulline precursors, such as ornithine or glutamine and/or are modified forms of citrulline (or of precursors thereof), for example in order to improve its stability. Suitable examples of such modifications include citrulline salts (or precursors thereof), citrulline complex forms and citrulline forms wherein the carboxyl group has been esterified or transformed to an amide group and/or which amine or amide group has been substituted, especially protected, for example alkylated, preferably methylated, arylated or acylated, preferably acetylated.

According to the present invention, the amine and carboxyl groups of citrulline (or of precursors thereof) may thus be present as salts. To be mentioned as suitable examples are inorganic or organic acid salts, such as oxalic acid, lactic acid, acetic acid, malic acid, citric acid, fumaric acid, phosphoric acid, HCl, HBr, p-toluene sulfonic acid. Also suitable are base salts such as soda, potash, calcium hydroxide, ammonium hydroxide, malates, or amine salts, such as for example, ethanol amine, di-ethanol amine, tri-ethanol amine, N-methylglucamine, tri-(hydroxymethyl)-methylamine or bis-cyclohexamine.

The following compounds, as well as their mixtures in whatever proportions, are especially suitable for the implementation of the present invention: D,L-citrulline, L-citrulline, D-citrulline malate, L-citrulline malate, L-citrulline monoacetate, L-citrulline hydrochloride, L-citrulline methylester, L-citrulline ethylester, L-citrulline-n-hexylester, L-citrulline (benzoylmethyl)ester, alpha-N-benzoyl-L-citrulline methylester, N-Boc-citrulline, N1-2,4-dinitrophenyl-D,L-citrulline. Citrulline or derivatives thereof, such as defined hereabove for example, may be commercially available or may be synthesized according to methods known from the persons skilled in the art. Salts may be bought or may be easily obtained by combining aminoacids with another acid or a base in solvents such as ethanol, or dioxane or dialkylether or tetrahydrofurane.

According to the present invention citrulline can also be derived from natural products such as plants (including fruits). In particular, it may be extracted from plants belonging to the cucurbitaceae family, such as especially cucumber, calabash, netted melon, bitter melon and watermelon. Depending on the various embodiments of the present invention, citrulline may then be in a pure form or in the form of a plant extract containing the same, especially as an extract of plants belonging to the cucurbitaceae family, such as cucumber, calabash and preferably melon (netted or bitter) and watermelon. When <<in a pure form>>, citrulline according to the present invention is intended to mean citrulline in a free form and/or in the form of at least a salt or derivative thereof, such as previously defined for example.

As used herein, a <<HMG-CoA reductase inhibitor>> is intended to mean a compound capable of slowing down or hindering the production of mevalonic acid from HMG co-Enzyme A, a decisive step of cholesterol biosynthesis. Such a compound will be able to act by (i) inhibiting the gene expression of HMG-CoA reductase, or (ii) by slowing down or hindering the enzymatic reaction catalyzed through the HMG-CoA reductase, that is to say, the conversion of HMG co-Enzyme A to mevalonic acid. Typically an inhibitor according to the invention will be able to prevent the substrate fixation to the enzyme active site by fixing itself in place (competitive inhibition), or to produce a deformation of the enzyme so that it becomes inactive (allosteric, either uncompetitive or non-competitive inhibition). Such compounds will present in the form of pure molecules or as more or less complex mixtures (including at least a HMG Co-A reductase inhibitor) and especially as plant extracts.

To be mentioned as suitable examples of competitive HMG-CoA reductase inhibitors that may be used herein are compounds belonging to the class of statins. As a common characteristic, statins share a chemical structure consisting of an opened or closed lactone ring, especially bound to a group having a more or less hydrophobic nature. In some embodiments, statins may be selected for example, from the group consisting of synthetic statins, amongst which atorvastatin, pravastatin, fluvastatin, rosuvastatin, lovastatin and simvastatin, as well as their mixtures in whatever proportions. This list is not limitative and all the other statins known from the person skilled in the art acting as inhibitors of the enzyme HMG-CoA reductase may also be used in the present invention. In a particular embodiment, the statin employed is atorvastatin. Other statins to be also suitably used in the present invention are statins of natural origin, as well as their mixtures in whatever proportions. Monacolins should be mentioned as suitable examples thereof. Monacolins, and especially monacolin K (or lovastatin) may be derived from rice red yeast, obtained through the fermentation of Monascus Purpureus yeast, with rice. Such statins will be for example suitably used in some embodiments of the present invention in a pure form or as red yeast rice extract containing the same (typically rice fermented with yeast Monascus Purpureus, dried and thereafter powdered). Preferably a red yeast rice extract according to the present invention is a standardized extract including from 1 to 4% monacolin K, more particularly from 1 to 3% monacolin K and typically including 2% monacolin K.

Other inhibitors of the HMG-CoA-reductase enzyme to be also suitably used in the present invention are for example policosanol (or polycosanol) and berberin. Policosanol is a combination of long-chain primary alcohols (such as octacosanol and triacosanol), with following formula: CH₃—(CH₂)ₙ—CH₂OH (n ranging from 24 to 34), obtained from waxes derived from plants such as sugar cane and yam, or beeswax. Berberin is an alkaloid derived from plants belonging to the berberidacae family. Policosanol like berberin can be used in some embodiments either in a pure form or as plant extracts containing the same.

According to the present invention, a plant extract is obtained through a solid-liquid extraction, optionally followed with purification steps. A solid-liquid extraction is defined as a method for separating components that are contained in a solid body through solubilization with a solvent, for example an aqueous or an alcoholic one. As used herein, a <<plant extract>> is also intended to mean plants (in whole or as parts) which are dried or dehydrated and ground or powdered. Such extracts may be obtained through pressing, with or without solvent extraction, expression, hydrodistillation, aqueous or alcoholic extraction, filtration, fermentation, maceration, this list being in no case limitative. The extracts of the invention may be in the form of juice, aromatic concentrate, oil, tisane, infusion, macerate, dry matter, wort, hydroalcoholic extract or lyophilizate, this list being here again not imitative.

Inhibitors of the HMG-CoA reductase enzyme and their mixtures in whatever proportions could be used as their salts for example, but with no limitation, their inorganic or organic acid salts, such as oxalic acid, lactic acid, acetic acid, citric acid, malic acid, fumaric acid, phosphonic acid, HCl, HBr, p-toluene sulfonic acid. Also suitable would be base salts such as sodium, potassium, calcium hydroxide, ammonium, malate, or amine salts, such as for example, ethanolamine, di-ethanolamine, tri-ethanolamine, N-methylglucamine, tri-(hydroxymethyl)-methylamine or bis-cyclohexamine.

As a rule, according to the present invention, citrulline, and/or the bioequivalents thereof as well as inhibitors of the HMG-CoA reductase enzyme could be used in the form of their physiologically acceptable salts. As used herein, <<physiologically acceptable salts>> are intended to mean compounds which have the desired pharmacologic or cosmetic activity of the parent compound, without generating any toxic activity upon administration.

The present invention relates to a combination product including as active substances, at least:

(i) an inhibitor of the HMG-CoA reductase enzyme, and

(ii) citrulline,

for use in the treatment of excess weight or obesity and/or the treatment of excess body fat.

In a particular embodiment, the combination product is used for reducing the body fat or preventing its appearance while preserving the lean body mass. Such a combination product will thus be preferably adapted to obese people or to people being overweight.

The present invention further relates to the use of a combination product including as active substances, at least:

(i) an inhibitor of the HMG-CoA reductase enzyme, and

citrulline, for preparing a medication aimed at treating excess weight or obesity and/or excess body fat.

The present invention especially aims at humans but could also be dedicated to all other mammals, in particular to pets, farmed animals or sport-related animals.

As defined in the present invention, a <<treatment>> includes both preventive and curative treatments. It also encompasses the treatment of patients having a risk of developing an excess body fat, and/or excess weight or obesity.

As used herein, <<treating excess weight or obesity>> is intended to mean that the administration of a combination product, such as defined herein, and with a dosage determined by the person skilled in the art, makes it possible to reduce the weight of the treated patients (mammal: human or animal). Such a product also enables to prevent weight gain and/or to stabilize a weight as low as possible, and/or to delay the development of excess weight or obesity, especially in patients having a risk of developing excess weight or obesity.

As used herein, <<treating excess body fat>> is intended to mean that the administration of a combination product such as defined herein, and with a dosage determined by the person skilled in the art, makes it possible to reduce the body fat of the treated patients (humans or animals). Such a product also enables to prevent the accumulation of body fat and/or to maintain a body fat as reduced as possible, or to delay the accumulation of body fat, especially in patients having a risk of developing excess weight or obesity and/or of developing an excess body fat of pathological nature (i.e. associated with a growing burden of disease), as is often the case with abdominal adiposity.

Preferably the body fat loss occurs without any loss of the lean body mass (with no muscular atrophy), the weight loss is thus essentially associated with a targeted body fat loss.

Patients having a risk of developing excess weight or obesity, or having risk factors of developing excess weight or obesity include for example people having an impaired energy balance (i.e.: the ratio between energy input and energy expenditure) resulting from high-calorie diets (i.e.: with a high content of fat and/or sugar) and/or from a sedentary lifestyle, people suffering from anxiety, stress or depression, from a hormonal imbalance or a familial susceptibility to obesity (genetic origins), or taking prescription drugs which can cause weight gain such as glucocorticoids or antidepressants.

Said association comprising at least a HMG-CoA reductase inhibitor and citrulline is also especially aimed at individuals suffering from glucose metabolism disorders. Indeed, the applicant did demonstrate in the present application that such an association also has a synergistic effect on the improvement in glucose tolerance. Such a synergistic effect is all the more surprising that, as far as the applicant knows, an effect of citrulline on glucose metabolism disorders could not be demonstrated so far. Moreover, the data to be found in literature about the effect of statins, and in particular atorvastatin, on metabolism disorders other than dyslipemias are highly controversial. Very newly published studies do indeed demonstrate antagonist metabolic effects of different types of statins and examine their large-scale use. Kok and al. publications (Atherosclerosis, 2011, 215(1):1-8) and Kopstapanos and al. publications (CurrVascPharmacol, 2011, 215(1):1-8) report in particular the conclusions of several clinical assays and emphasize the fact that some statins have no effect on insulin sensitivity, even would increase insulin resistance and report notably that atorvastatin, rosuvastatin and simvastatin would promote the development of type 2 diabetes. Experiment results suggest moreover that the lipophilic character of some statins, as well as the inhibition of the enzyme HMG-CoA-reductase, could be factors responsible for a negative effect of statins on the glucose metabolism (Kopstapanos and al., CurrVascPharmacol, 2011, 215(1):1-8).

A combination product comprising the active substances (i) and (ii) in any of the forms defined in the present application will thus be used for treating and/or preventing glucose metabolism disorders, in particular for treating glucose intolerance or insulin resistance.

Since it could be shown that the combination of the present invention preserves the lean body mass, the use of such a combination will be especially recommended for elderly people suffering from glucose intolerance and/or insulin resistance. Indeed, such patients have especially a risk of suffering from or developing a lean body mass loss (muscle mass) and/or a protein-energy undernutrition associated with restrictive diets (which by the way are not allowed for elderly people according to the HAS recommendations (French high authority on health) (Raynaud-Simon and al., 2007).

As used herein, <<elderly people>> are intended to mean patients aged of 65 or more, preferably patients aged of 70 or more and especially patients being at least 75 years old. According to the present invention, glucose metabolism disorders (Impaired Glucose Metabolism, IGM) are defined as being blood glucose levels above the normal but lower than those stated for diagnosing type 2 diabetes II, or diabetes mellitus. The prevalence of glucose metabolism disorders varies from country to country but is generally 2 to 3 times higher than for diabetes. Until recently, subjects suffering from glucose metabolism disorders were considered as prediabetics, but the results of epidemiologic studies demonstrated that those subjects were in fact heterogeneous as regards the risks of developing diabetes and/or cardiovascular diseases.

Amongst the subjects suffering from glucose metabolism disorders, about 58% are glucose intolerant (Impaired Glucose Tolerance, IGT), 29% have a moderate fasting hyperglycemia (Impaired Fasting Glycemia, IFG) and 13% have both disorders (IGT/IFG). Said association according to the present invention is thus aimed at individuals having either one or both glucose metabolism disorders.

IGT is characterized by a high postprandial hyperglycemia. IGT measurement method is standardized with the oral glucose tolerance test (OGTT), measured two hours after ingestion of a glucose dose (1.75 g glucose per kg, typically 75 g for adult males). In healthy subjects, the level is normally under 7.8 mmol/l (140 mg/dl). Levels ranging from 7.8 to 11.1 mmol/l (140 to 200 mg/dl) are an indication of glucose intolerance. Glucose levels that would be above the latter threshold do confirm a diagnosis of diabetes. In people in good health, fasting glycemia is traditionally under 6.1 mmol/l (110 mg/dl). IFG is defined by fasting blood sugar levels ranging from 6.1 to 7 mmol/l (110 to 125 mg/dl). Above 7.0 mm/l (126 mg/dl), fasting glycemia does illustrate a diabetic condition.

According to the present invention, and unless otherwise specified, the expressions <<comprised between>> and <<from ( . . . ) to >> should be understood as including the limits of the interval.

A combination product of the invention may have many different forms. As a rule, it is provided according to the present invention as:
(1) a composition comprising at least as active substances (i) a HMG-CoA reductase inhibitor, and (ii) citrulline, in a physiologically acceptable medium.
   Such a composition is also referred to as a single composition (or preparation) (or combined composition).
(2) A kit comprising as active substances:
(i) at least a HMG-CoA reductase inhibitor, optionally in a physiologically acceptable medium; and
(ii) at least citrulline, optionally in a physiologically acceptable medium;
   said active substances (i) and (ii) each being provided in a form that is adapted to a joint administration of both substances.

As used herein, a <<kit>> is intended to be a synonym of box or set and corresponds to a group of products that are presented together.

Preferably, the active substances (i) and (ii) may be respectively in the form of compositions (a) and (b) containing the same at least, and wherein said active substances (i) and (ii) are in a physiologically acceptable medium.

According to a further aspect of the present invention, it is provided a method for preparing a kit, such as previously defined, said method comprising a step of conditioning the active substance (i) and the active substance (ii), thus making both active substances adapted to a joint administration.

The compositions (a) and (b) such as previously defined may be:
(i) in the form of distinct compositions (i.e.: independent from each other), later on associated to be used in conjunction with each other, as part of a combined treatment;
(ii) presented together in a same container in the form of distinct compositions in a joint packaging to be used in conjunction with each other, as part of a combined treatment.

A kit according to the present invention may also contain use instructions for the components included therein.

As referred to the combination products as a kit, the <<joint administration>> according to the present invention is intended to mean that the active substances (i) and (ii) or the compositions (a) and (b) comprising respectively at least (i) a HMG-CoA reductase inhibitor, in admixture with a physiologically acceptable medium and (ii) citrulline, in admixture with a physiologically acceptable medium, are administered sequentially (i.e.: successively, in a staggered manner over time), separately and/or simultaneously during the treatment period.

Referring to the combination products according to the present invention, the <<joint administration>>, is intended to mean that the active substances (i) and (ii) of the combination product are administered (optionally repeatedly) either together, either separately, within a short time enough to enable a positive synergistic effect between the two components, such as previously defined. How to obtain such a synergistic effect will especially depend on the expected effect and/or on the patient, and/or on the formulation of the components.

The compositions according to the present invention may thus be administered simultaneously, as a single composition or as many distinct compositions. Should the compositions be distinct from each other, they also may be administered sequentially, for example at different times of the day, (indeed a composition comprising at least one of the active substances (i) or (ii) could be administered in the morning and the other one could be administered at noon) and/or at daily frequencies.

As used herein, <<in conjunction with each other>> is intended to mean that either of both compositions (a) or (b) may be administered (optionally repeatedly or continuously) before, after, and/or at the same time as the administration of the other one composition. It is for example possible for each of the compositions (a) and (b) to be administered at different times of the day, or that one of the two compositions (a) or (b) be taken at a different frequency as compared to the other. As an example, a composition (a) comprising at least a HMG-CoA reductase inhibitor could be administered every day, once a day, and a composition (b) comprising at least citrulline could be taken every day once to three times a day.

The formulation of the combination products according to the present invention as well as the nature and the amount of the active substances in the single composition or in the compositions of the kit according to the present invention depend on the therapeutic or cosmetic purpose of the treatment.

The active substances (i) and (ii) of the respective compositions (a) and (b) may be in physiologically acceptable media having a same or a different nature. A physiologically acceptable medium is a medium which has no toxic or detrimental effect in the conditions of use and which is inert towards the active substances of the combination product. Such a medium may for example comprise various additives depending on its therapeutic or cosmetic purpose, such as, without limitation, flavors, coloring agents, fillers, preservatives, diluents, wetting agents or suspending agents, etc. Such media may also enable an immediate release, a modified release or a controlled release of one of both active substances, whatever their presentation, either together as a single composition or separately as a kit.

In some embodiments of the present invention, a combination product of the invention may comprise, in addition to the active substances (i) and (ii), respectively the HMG-CoA reductase inhibitor and citrulline, other additional active substances or compounds, provided said additional active substances or compounds are not detrimental to the therapeutic or cosmetic activity of said association of the invention. Said additional active substances may be added to the active substances (i) and (ii) in the event of a single composition or to one and/or to the other of compositions (a) and (b) in the event of a kit.

Advantageously, an additional active substance stimulates the weight loss or the body fat reduction and/or contributes to the prevention of weight gain or of body fat development and/or promotes the improvement in glucose tolerance.

Referring to the pharmaceutical forms (i.e.: with a therapeutic purpose) of the present invention, other active substances may also be lipid-lowering agents such as cholesterol intestinal absorption inhibitors or fibrates, drugs improving insulin sensitivity or stimulating insulin production, such as drugs belonging to the biguanide family, glitazones, hypoglycemic agents such as sulfamides.

Any additional compound with a nutritional interest and/or stimulating weight loss or body fat reduction or having positive properties as to the metabolism of cholesterol, of lipids and/or of glucose may also be added to the combination products of the present invention in a pharmaceutical or cosmetic form. The combination product could comprise, for example, vitamins, mineral salts, essential amino acids, essential fatty acids, oligo-elements, various plant extracts, fibers, antioxidants, flavonoids. A combination product of the invention could in particular comprise natural components having lipid-lowering properties, such as guggul gum or prickly pear extracts (nopal), anorectic properties, such as hoodia extracts, eucalyptus extracts, laurel leaf extracts, coca leaf extracts, catharanthus extracts, phyllantus niruri extracts, orthosiphon extracts, algae extracts, carob extracts, konjac extracts, fat-burning properties (or increasing thermogenesis), such as fucus extracts, seaweed extracts, green tea extracts, mate extracts, guarana extracts, coleus extracts, garcinia extracts, caffein extracts, purgative properties, such as dandelion extracts, artichoke extracts, rosemary extracts, milk thistle extracts, turmeric extracts, hercampuri extracts (gentianella) or tranquilizing properties to reduce the stress frequently involved in obesity and to allow to better tolerate the hypocaloric diet, such as St Johns wort extracts (Hypericum), California poppy extracts (Eschscholtzia), valerian extracts (Valeriana), linden extracts (Tilia).

Suitable pharmaceutical or cosmetic formulations may be commercially available, or be described in literature, as for example pharmaceutical formulations, in Remington—The Science and Practice of Pharmacy, 19th ed., MAck Printing Company, Easton, Pa. (1995) and in Martindale—The Complete Drug Reference (34th edition) as well as the documents incorporated by reference. Finally, the preparation of suitable formulations and in particular of a single composition comprising the active substances (i) and (ii) may be conducted by the person skilled in the art according to routine methods.

It is understood that the various active substances of a kit according to the present invention may present in pharmaceutical or cosmetic forms which are the same or different and which may be administered using the same or different routes.

The single composition or the distinct compositions of a kit according to the present invention are preferably locally or systemically administered, for example using the following oral, enteral, sublingual, intravenous or intra-arterial, intramuscular, cutaneous, subcutaneous, transmucosal, rectal, transdermal, nasal, pulmonary, topic routes as well as through any parenteral route, in a physiologically acceptable dosage form. Preferred routes of administration are enteral, in particular oral, and intravenous routes. As an example, composition (a) could be administered intravenously

(i.v.). continuously or by bolus, and composition (b) could be administered every day by mouth.

For enteral, oral, sublingual, parenteral, subcutaneous, intramuscular, intravenous, or intra-arterial, nasal, pulmonary, topic, transdermal, local or rectal administration, the compositions of the present invention may be administered to humans or animals as unit doses in admixture with physiologically acceptable media known from the person skilled in the art.

Preferably, a combination product of the invention is administered by mouth. Suitable forms for oral route are for example tablets, hard gelatin capsules, lozenges, powders, granules, lyophilizates, oral solutions and syrups. Tablets, powders, granules, lyophilizates, oral solutions and syrups represent the currently most preferred pharmaceutical or cosmetic form adapted to oral administration. If a solid composition in the form of tablets should be prepared, it would be possible for example to combine the main active substance (i) or (ii) with a physiologically acceptable vehicle such as gelatin, starch, lactose, magnesium stearate, talc, gum arabic or equivalents. Tablets may be of various types, providing an immediate release, or a controlled or a slow release, and optionally be in an effervescent or oro-dispersible form. Gelatin capsules may be obtained by combining the active substance (i) or (ii) with a diluent and pouring the resulting mixture into soft or hard capsules.

A preparation in the form of syrup or elixir may for example contain the active substance (i) or (ii) together with a suitable sweetener, antiseptic agent, preservative, flavoring agent or coloring agent.

Powders, lyophilizates or granules that are dispersible in water may contain the active substance (i) or (ii) in admixture with dispersing agents or wetting agents or suspending agents, as well as with taste modifiers or sweeteners.

As an example, composition (a) including at least as an active substance (i), a HMG-CoA reductase inhibitor could be in the form of capsule or tablet and composition (b) including at least as an active substance (ii), citrulline could be in the form of capsule, powder, lyophilizate, granules or tablet but also in a drinkable form, such as a syrup, an infusion, a juice, a macerate, this list being in no case imitative.

In a particularly preferred embodiment, compositions (a) and (b) come as unit doses, of the capsule, tablet or sachet type. Such a sachet may comprise for example some powder, a lyophilizate or granules. Most preferably, composition (a) comes in the form of tablet and composition (b) in a sachet unit form.

The combination products according to the present invention in a suitable form to be administered by mouth may also be used as food supplements. In such an embodiment, the active substances (i) and (ii) in the form of a single composition or in the form of separate compositions (kit) are in a medium adapted to oral administration. The active substances (i) and (ii) may also be directly mixed with a dietary medium, food or beverage.

When the combination product of the invention is in a form that is adapted to oral administration in admixture with food or beverage, it can be considered as a functional food. As used herein, a <<functional food>> is intended to mean any food or beverage, for example, milk products from animal origin (butter, cheese, yogurt, milk) or from vegetable origin (milk, yogurt, cereals, fruit or vegetable juices, soups, etc.) to which a combination product of the invention has been added. Preferably, said combination product then comes as a single composition added to food, as previously mentioned.

A combination product, for use as a food supplement or functional food such as described hereabove can of course also comprise any additional compound having a nutritional interest and/or stimulating weight loss or body fat reduction, such as vitamins, mineral salts, essential amino acids, essential fatty acids, oligo-elements, various plant extracts, fibers, antioxidants, flavonoids.

When the combination product of the invention is for use as a food supplement, it can also comprise components, and in particular the active substances (i) and (ii), as plant extracts containing the same, such as previously described.

The present invention thus further relates to a combination product comprising as active substances at least:

(i) a HMG-CoA reductase inhibitor in the form of a plant extract containing the same, preferably a red yeast rice extract, and/or a plant extract including monacolins and/or berberin and/or policosanols;

(ii) citrulline, preferably in the form of a plant extract containing the same,

said substances (i) and (ii) being in a form adapted to a sequential, a separate and/or a simultaneous oral administration with one another over time.

In a particular preferred embodiment, the plant extract including citrulline and/or one of the bioequivalents thereof is a plant extract obtained from a plant belonging to the cucurbitaceae family, in particular a watermelon and/or a melon extract.

In another embodiment, the combination product comprises as active substances:

(i) a HMG-CoA reductase inhibitor in the form of a plant extract containing the same, preferably a red yeast rice extract, and/or a plant extract including monacolins and/or berberin and/or policosanols;

(ii) citrulline, in a pure form,

said substances (i) and (ii) being in a form adapted to a sequential, a separate and/or a simultaneous oral administration with one another over time.

In a particular embodiment, the combination product comprises active substances consisting in:

(i) at least a HMG-CoA reductase inhibitor in the form of a plant extract containing the same, preferably a red yeast rice extract, and/or a plant extract including monacolins and/or berberin and/or policosanols;

(ii) citrulline, in a pure form,

said substances (i) and (ii) being in a form adapted to a sequential, a separate and/or a simultaneous oral administration with one another over time.

Preferably such combination products are adapted to the oral route, and still preferably they are to be used as food supplements.

The amount of active substances (i) and (ii) in the products of the present invention as well as the dosage of said products will depend on the cosmetic or therapeutic purpose of the present invention. In particular for a therapeutic treatment, this amount will vary depending on the disorder to be treated and on the severity of the patient's condition, on the individual as such, on the administration route, as well as on the form of compounds (i) and (ii).

The dosage administered to a mammal, human or animal, in the framework of the present invention must be sufficient to induce a therapeutic or a cosmetic effect within a reasonable time interval. The person skilled in the art will determinate the right dosage, the composition and the administration route that are the best suited to a combination product according to the present invention. In particular for a pharmaceutical treatment, those can vary depending on the pharmacological properties of the formulation, on the nature and the severity of the disorder to be treated (and/or the excess weight or obesity), on the physical condition of the patient or individual, on the efficiency of the selected active substances (i) and (ii) as well as on age, weight, sex and on the response of the patient to the treatment.

The administration of the active substances (i) and (ii) according to the present invention may be continuous or intermittent (bolus). The dosage may thus be determined based on the schedule and/or the frequency of administration. Suitable dosages of active substances comprise those which can be found in the medical literature such as in Martindale—The Complete Drug Reference (34th edition) as well as the documents included by reference. Moreover, adapting the dosage from a species to one another belongs to the competencies of the person skilled in the art and may be conducted such as described in Mordenti and al., Pharmarceut. Res. 8, p. 1351-1358, 1991. For example suitable dosages of active substances vary from about 0.01 mg/kg body weight to 1000 mg/kg body weight. Preferably, dosage ranges from 0.1 mg/kg to 20 mg/kg per day and for oral administration.

As an example, the active substance (i) of a combination product of the invention is administered according to a daily dosage ranging from 10 to 80 mg per day and preferably at a daily dose ranging from 30 to 50 mg, for synthetic statin, for example atorvastatin, simvastatin or pravastatin. When the active substance (i) is a natural statin, in the form of a standardized rice red yeast extract containing the same, said extract is typically administered at doses of from 100 to 600 mg one to three times a day. Preferably such a standardized extract comprises from 1 to 4% monacolin K, more particularly from 1 to 3% and typically such an extract contains 2% monacolin K.

Citrulline is typically administered at a daily dose ranging from 1 to 15 g/day and preferably at a dose of 2 to 5 g/day. These doses could be administered all at once or being divided, for example in two or three times. The person skilled in the art will adapt the dosage of the compositions of the present invention based on the pharmaceutical form and on the number of doses taken, in accordance with the administration of the desired active substance dose.

Thus, when the compositions of the invention come as unit doses such as previously described, they may contain citrulline doses ranging from 50 to 4000 mg, in particular from 100 to 3000 mg, in particular from 200 to 1000 mg. Unit doses may also comprise statin doses ranging from 2 to 100 mg, from 5 to 80 mg or from 10 to 40 mg. Last, red yeast rice can be present in unit doses of the present invention ranging from 30 to 1000 mg, from 50 to 800 mg or from 100 to 600 mg.

For example, when the combination product comes as a single composition, said composition could be administered by mouth or in a continuous manner per intravenous infusion. Such an administration may be effected for some time depending on the patient and his condition. The administration could be interrupted, and then repeated one or many times. However, such a composition is preferably adapted to oral administration.

When the combination product comes as a kit including at least a composition (a) and a composition (b) both distinct, such as previously described, it is intended to mean that both compositions could be administered using the same or similar routes. For example a composition could be injected, for example continuously per infusion and another composition of the combination product could be administered enterally, in particular per mouth, bolus or be divided in many times, during the day. Both compositions may also be administered enterally, in particular per mouth, at the same or different frequencies. Preferably the compositions (a) and (b) are administered by mouth one or more times a day, a composition being taken per bolus every day, for example in the morning or in the evening, and the other one be divided in many times during the day, for example, in the morning and evening or in the morning, at noon and in the evening. When the active substances (i) and (ii) are present in two distinct compositions (a) and (b), it is possible to take said substances at different frequencies or duration, or to take them sequentially (one cure after the other or simply at different times of the day). For example the substance (i) may be taken once a day whereas the substance (ii) could be taken 3 times a day. Substance (ii) may also be prolonged for a time period after stopping the cure with substance (i).

The administration of a combination product such as described in the present invention can thus be conducted repeatedly, in the form of a cure lasting a few days or weeks (for example 4 weeks). Optionally, several successive cures may be carried out, for example 2 to 4, optionally interrupted with a break of 1 week to 3 months. As part of a preventive treatment, the administration of a combination product of the invention may be conducted chronically.

The present invention further relates to a method for treating weight severe disorders, such as excess weight or obesity, and excess body fat, comprising the administration of a combination product such as previously described and its optional renewal until the desired therapeutic effect is obtained.

The present invention further relates to a method for treating excess weight or obesity and/or excess body fat, comprising the administration (i) of at least an inhibitor of the HMG-CoA-reductase enzyme, and (i) citrulline. In a particular embodiment, said method may comprise the administration of at least one additional active substance for the same therapeutic indication and/or for complementary indications as for example for the treatment of dyslipemias or for the treatment or prevention of cardiovascular disorders. In a preferred embodiment, the active substances (i) and (ii) are the only substances administered as active substances for treating excess weight or obesity and/or excess body fat.

The present invention finally relates to a cosmetic treatment method for improving the appearance of individuals, for example for slimming the figure and/or reducing or restraining localized fat accumulations or lipodystrophia, and/or for stimulating the loss of excess weight and/or of cellulite, and/or for limiting the accumulation thereof, preferably without any loss of the lean body mass in individuals who are in good health.

As used herein, treating <<excess weight>> is intended to mean that said cosmetic method is aimed at subjects (humans or animals, for example pets, farmed animals, sport-related animals or animals dedicated to shows) with a normal corpulence. In humans for example, people will be considered, who are in good health, the excess weight or fat accumulation of whom is not associated with pathological conditions or a growing burden of disease (typically glucose metabolism disorders, insulin resistance, metabolic syndrome, diabetes or vascular disorders) and the BMI of whom is from 18.5 to 25. In these subjects, excess weight or body fat typically exist as cellulite, is not associated with a growing burden of disease and is preferably distributed according to a gynoid pattern. A cosmetic treatment according to the present invention will therefore be especially suited to the treatment of cellulite, in particular on hips and buttock. It could be used for example in women who go through menopause or after pregnancy and, as a rule, in individuals having a sedentary lifestyle and/or a diet that is high in sugar or in fat, so as to contribute to preserve or to rediscover a thin figure and to remove or prevent the development of cellulite. Such a treatment may also be useful in castrated animals so as to prevent the increase in body fat at the cost of muscular lean mass.

Weight loss is often induced through restrictive diets, that are hard to observe in the long term and that are frequently responsible for a so called rebound effect (or relapse) with, as a consequence, a new weight gain sometimes higher than the one induced through the caloric restriction itself. A cosmetic treatment method according to the present invention could thus be used as a complement to a restrictive diet while optionally increasing physical activity, so as to limit the intensity of the restriction, especially the caloric one, and so as to prevent any lean body mass loss and rebound effect when stopping the restrictive diet.

A cosmetic treatment method according to the present invention comprises the administration of at least (i) a HMG-CoA reductase inhibitor and (ii) citrulline, in particular in the form of a combination product such as previously defined. Such a combination product may be in the form of a single composition or as a kit, such as previously described.

In such a method, the administration of at least (i) a HMG-CoA reductase inhibitor and (ii) citrulline and/or one of the bioequivalents thereof could be conducted simultaneously as a single composition or as many separate compositions. When the compositions are distinct from each other, they may also be administered separately and/or sequentially over time, according to the previously defined modalities.

Most preferably, the combination product is adapted to oral administration and is intended to be taken as a food supplement such as previously described. Said product could of course comprise any additional compound with a nutritional interest and/or stimulating weight loss or body fat reduction such as previously described. The combination product could comprise, for example, vitamins, mineral salts, essential amino acids, essential fatty acids, oligo-elements, various plant extracts, fibers, antioxidants, flavonoids. Natural components may also be mentioned which have anorectic properties, such as hoodia extracts, eucalyptus extracts, laurel leaf extracts, coca leaf extracts, catharanthus extracts, phyllantus niruri extracts, orthosiphon stamineus leaf extracts, algae extracts, carob extracts, konjac extracts, fat-burning properties (or increasing thermogenesis), such as fucus extracts, seaweed extracts, green tea extracts, mate extracts, guarana extracts, coleus extracts, garcinia extracts, caffein extracts, purgative properties, such as dandelion extracts, artichoke extracts, rosemary extracts, milk thistle extracts, turmeric extracts, hercampuri extracts (gentianella) or tranquilizing properties to reduce the stress frequently involved in obesity and to allow to better tolerate the hypocaloric diet, such as St John's wort extracts (Hypericum), California poppy extracts (Eschscholtzia), valerian extracts (Valeriana), linden extracts (Tilia).

In a particular embodiment, the active substances (i) and (ii) are the only substances administered as active substances for slimming the figure and/or reducing or restraining localized fat accumulations or lipodystrophia, and/or for stimulating the loss of excess weight and/or of cellulite, and/or for limiting the accumulation thereof. In another embodiment, the active substances (i) and (ii) are the only substances administered as active substances.

Moreover, such a combination product preferably comprises components, and in particular the active substances (i) and (ii), of natural origin, typically as plant extracts. For example the combination product may comprise (i) a red yeast rice extract, and/or a plant extract including policosanol, and/or berberin and/or monacolins and (ii) a plant extract obtained from plants of the cucurbitaceae family, in particular a watermelon and/or a melon extract.

For men, a standardized red yeast rice extract is typically administered at doses ranging from 100 to 600 mg one to three times a day. For men, citrulline is typically administered at doses ranging from 1 to 15 g/day, in particular at doses ranging from 2 to 5 g/day.

The present invention will be hereafter illustrated without being limited thereto by means of the following examples. It will be referred to the following figures in the examples:

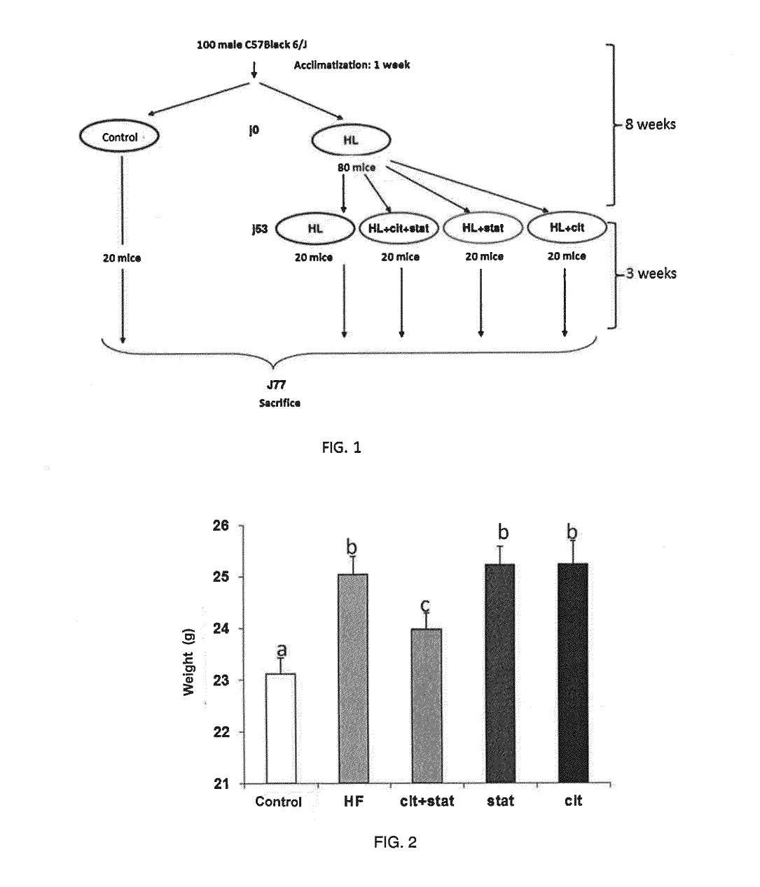

FIG. 1. Schematic representation of the time sequences of the implemented pharmaco-nutritional experiments. (HL): high-lipid diet, (HL+cit): high-lipid diet supplemented with citrulline only, (HL+cit+stat): high-lipid diet supplemented with citrulline and atorvastatin, (HL+stat): high-lipid diet supplemented with atorvastatin only

FIG. 2. Effects of the various diets: control, HL, cit+stat (HL+cit+stat), stat (HL+stat), cit (HL+cit), on mice weight. a vs. b and c vs. B: p<0.05; a vs. c=0.06.

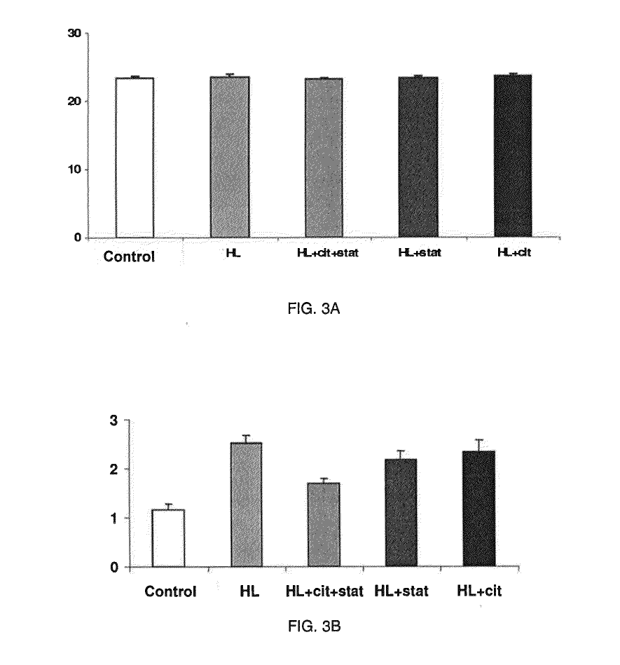

FIG. 3. Effects of the various diets: control, (HL), (HL+cit), (HL+cit+stat) and (HL+stat), on mice lean body mass (FIG. 3A) and on mice body fat (FIG. 3B) tested using EchoMRI.

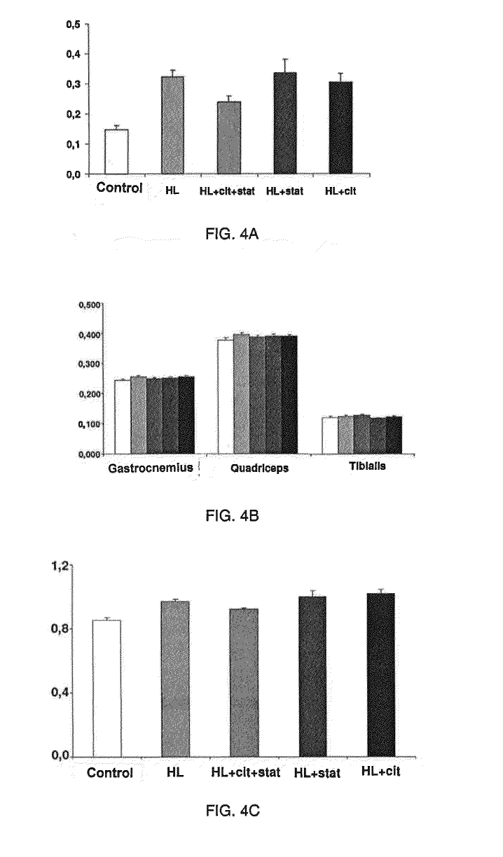

FIG. 4. Effects of the various diets: control, (HL), (HL+cit), (HL+cit+stat) and (HL+stat), on adipose tissue weight (FIG. 4A), leg muscles (FIG. 4B) and liver (FIG. 4C).

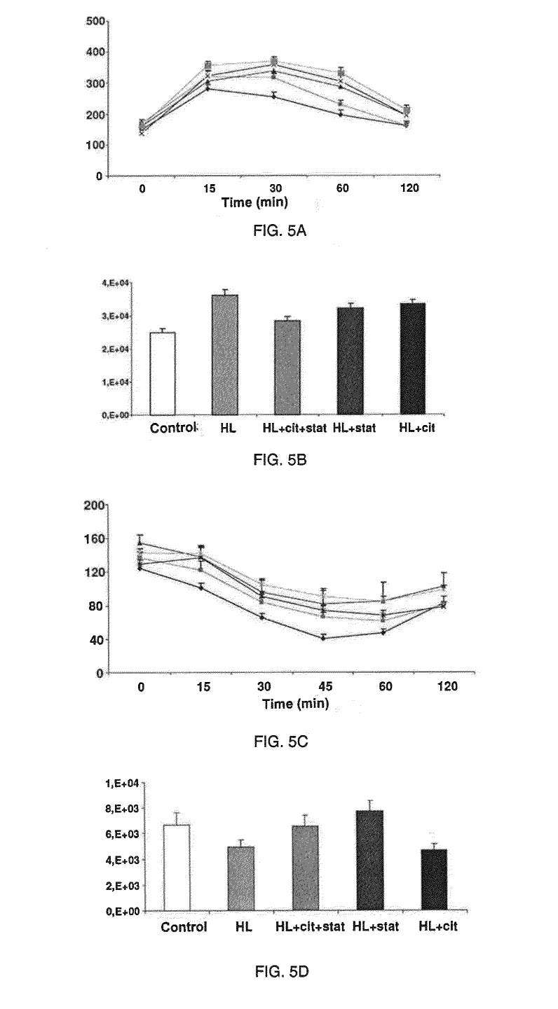

FIG. 5. Effects of the various diets: control (♦), HL (▪), HL+cit (x), HL+cit+stat (▪) and HL+stat (▴), on glucose tolerance (FIG. 5A: glycemia (mg/dl), FIG. 5B: Glucose AUC (i.e.: area under the curve, arbitrary unit) and on insulin sensitivity (FIG. 5C: glycemia (mg/dl), control (♦), HL (▪), HL+cit (x), HL+cit+stat (x) and HL+stat (▴)), FIG. 5D: glycemia decrease (UA)).

### EXAMPLES

### 1. Materials and Methods

### 1.1 Animals:

100 male mice C57 Black 6/J aged 5 weeks (January, France) were accommodated in individual cages in the pet center of the INRA center, Theix, in thermoregulated premises (20-22° C.) and submitted to alternating 12 hour-cycles of light and darkness. After a week of acclimatization under a <<control regimen>> (D12450B, Research Diet, USA), 20 mice continued this regimen for 11 weeks. The other 80 mice were fed with a [symbol] high-lipid regimen [symbol] (HL) (D12451, Research Diet, USA) for 8 weeks. The total calorie intake, in percentages, provided for both the <<control>> and HL regimens 20% proteins and respectively, 70% and 35% carbohydrates and 10% and 45% lipids. After height weeks under the high-lipid diet, the 80 mice were distributed into 4 groups of 20 animals and respectively submitted to a high-lipid diet alone (HL), enriched with L-Citrulline only (2.5 g/kg) (HL+cit), Atorvastatin only (10 mg/kg) (HL+stat) or L-Citrulline (2.5 g/kg) and Atorvastatin (10 mg/kg) (HL+cit+stat), for three weeks. The animals all had a free access to food and water for the all duration of the study (FIG. 1). The mice weight was measured every 2 weeks.

### 1.2 Insulin Sensitivity and Glucose Tolerance Measurements

After a 3-week pharmaco-nutritional regimen, measurements of the insulin sensitivity and glucose tolerance were conducted respectively on 10 mice of each group after a fasting night. Insulin sensitivity measurement: basal glycemia was evaluated using a glucose meter (Glucotouch, Lifescan, USA) on blood droplets collected from the tail. An insulin dose of 0.75 mU/g of weight was administered through intraperitoneal injection and glycemia was evaluated 15, 30, 45, 60 and 120 minutes post injection.
Glucose tolerance measurement: a glucose dose (2 mg/g of weight) was administered through intraperitoneal injection. Glycemia was evaluated as for the insulin sensitivity test before and 15, 30, 60 and 120 minutes post injection.

### 1.3 Body Composition Measurement Through ECHO MRI

Body fat and lean body mass of awake mice after 15 h fasting were quantified using nuclear magnetic resonance (Echo MRI, Houston, USA) at day 80 of the experiment.

### 1.4 Organ and Tissue Collection

Prior to being sacrificed, mice after 15 h fasting were anesthetized through barbital intraperitoneal injection (1.5 μl/g). Blood was collected in EDTA-containing specimen collectors before being centrifuged (12000 rpm, 4 min, room temperature). Plasma was thereafter collected, distributed in aliquots and frozen in liquid nitrogen, then stored at −80° C. The adipose peri-epididymal tissue, liver and leg triceps muscle (gastrocnemius muscle), tibialis and quadriceps muscles were weighted, frozen in liquid nitrogen, then stored at −80° C.

### 1.5 Plasma Dosages

Plasma levels of non-esterified fatty acids (AGNE) (NEFA-HR(2) R1 and R2 434-91795/436-91995, Wako Chemicals GmbH), triglycerides (TG) (981786, Thermo Fischer Scientific), cholesterol (981813 Thermo Fischer Scientific), and glucose (Glucose God-Pod, 981780 Thermo Fischer Scientific) were measured using the automated device Konelab 20 (Thermo Electron Corporation, France).

### 1.6 Statistic Analysis

The results were expressed as mean±standard error of the mean. The results were analyzed through a variance analysis (ANOVA) and a Fischer post-hoc test using the software Statview (SAS Institute, USA). The significance level was fixed to p<0.05.

### 2. Results

### 2.1 Animal Weight Evolution

After a 3-week pharmaco-nutritional regimen, the weights of mice from the various groups were significantly different (FIG. 2, ANOVA, p<0.0001). The weights of the mice submitted to the HL, HL+stat and HL+cit regimens were significantly higher as compared to the controls (respectively, p<0.001, p<0.0001, p<0.0001). Only HL+cit+stat regimen revealed capable of significantly reducing the mice weight as compared to the other mice submitted to HL, HL+cit or HL+stat regimens (P<0.05) and at a level close to that of controls (p=0.06).

### 2.2 Animal Body Composition Analysis

Echo MRI Analysis: mice lean masses of the 5 groups were not significantly different (FIG. 3A, ANOVA, p=NS). By contrast, total body fat of the mice submitted to HL, HL+cit, HL+stat and HL+cit+stat regimens was significantly higher (respectively +115%, +100%, +87%, +46%) than that of the mice having followed the control regimen (ANOVA, p<0.0001). However, total body fat of the mice submitted to HL+cit+stat regimen was lower than that of HL (−32%), HL+cit (−27%) and HL+stat regimens (−22%)(P=0.06) (FIG. 3B).

Upon weighting tissues when sacrificing: mice having followed HL, HL+cit, HL+stat regimens had doubled the weight of their peri-epididymal adipose tissue as compared to mice under control regimen (FIG. 4A, ANOVA, p<0.005). The weight of peri-epididymal adipose tissue of mice having followed HL+cit+stat regimen was lower as compared to that of the other groups HL (−26% vs. HL, −22% vs. HL+citr, −30% vs. HL+stat) (FIG. 4A). The absence of significant difference between mice lean masses analyzed through Echo MRI was confirmed after weighting the organs. The addition of all leg triceps, tibialis and quadriceps muscle weights was not significantly different from one group to another (FIG. 4B). Liver weight did not significantly vary either (FIG. 4C).

### 2.3 Plasma Parameters

Plasma levels of non-esterified fatty acids, triglycerides, cholesterol and glucose for the various groups are shown in Table 1. Plasma levels of non-esterified fatty acids in mice submitted to HL regimen were significantly higher, on average by 30% higher than those of the other groups (ANOVA, p<0.05). HL+cit, HL+cit+stat regimens and, to a lesser extent, HL+stat, could establish plasma levels of non-esterified fatty acids at a similar level compared to the control mice. Similarly, triglyceride plasma levels were significantly higher in the group of the mice submitted to HL regimen as compared to mice having received the other regimens (ANOVA, p<0.05). HL+cit, HL+stat and HL+cit+stat regimens could bring triglyceridemia back to the level of that obtained in control mice. HL, HL+cit, HL+stat and HL+cit+stat regimens induced a significant increase in cholesterolemia as compared to that of the control mice (ANOVA, p=0.01). No significant difference in glycemia could be evidenced amongst the 5 groups (Table 1).

### 2.4 Insulin Sensitivity and Glucose Tolerance

Glucose tolerance measurement (ipGTT test): glucose tolerance is defined as the ability for a mouse to normalize its glycemia when injecting glucose. It is evaluated through the calculation of the area under the curve of the glycemia evolution after an injection of glucose.

HL, HL+cit, HL+stat regimens produced a significant reduction of glucose tolerance as compared to the control regimen (respectively p<0.0001; p=0.0001 and p=0.0008). Only HL+cit+stat regimen could bring the glucose tolerance back to that of control mice. The glycemic response of this HL+cit+stat group was improved as compared to that of the HL+stat group (p=0.07) and was significantly different from those of the HL+cit and HL groups (respectively p<0.05 and p<0.001) (FIGS. 5A and 5B).

Insulin sensitivity measurement (ipITT test): similarly, the mice under HL or HL+cit regimen tend to be less sensitive to insulin as compared to controls (respectively, p=0.10 p=0.07) and to HL+stat group (respectively p<0.05 and p<0.01). HL+cit+stat regimen could maintain insulin sensitivity to a level comparable to that of the control mice (FIGS. 5C and 5D).

### 3. Conclusion

Said association of L-citrulline (whether of natural or synthetic origin) and statin (whether of natural or synthetic origin) makes it possible to limit weight gain and produces a strong synergistic effect on the body fat gain, under obesogenic regimen.

Moreover, such an association also has a synergistic effect contributing to the prevention of glucose intolerance and insulin resistance associated with excess weight gain.

Table 1 hereunder illustrates the synergistic actions between L-citrulline and statin for the prevention of weight gain and metabolic syndrome resulting from high-lipid diet:

TABLE 1

| | | |
|---|---|---|
| L-citrulline | Statin | L-citrulline + statin |

Weight and body composition

| | | | |
|---|---|---|---|
| Body weight | — | — | −4% |
| Body fat | −7% | −13% | −32% |

Plasma levels

| | | | |
|---|---|---|---|
| Glucose | — | — | — |
| Triglycerides | −15% | −13% | −15% |
| Non esterified fatty | −24% | −11% | −17% |
| acids | | | |
| Cholesterol* | — | — | — |

Glucidic homeostasis

| | | | |
|---|---|---|---|
| Glucose tolerance | +7% | +11% | +21% |
| Insulin sensitivity | +5% | +31% | +34% |

Percentages illustrate the differences with high-lipid diet alone (HL); “—” stands for a non significant effect (NS).
*The mouse is not a good model to explore the LDL/HDL response.

1. A combination product including as active substances, at least:
i. an inhibitor of the HMG-CoA-reductase enzyme, and
ii. citrulline;
for use in the treatment of excess weight or obesity, or excess body fat;
said product being further characterized in that citrulline is administered at a daily dose of from 1 to 15 g/day.

2. A combination product for use according to claim 1, characterized in that the active substances consist in:
i. at least an inhibitor of the HMG-CoA-reductase enzyme, and
ii. citrulline.

3. A combination product for use according to claim 1, characterized in that it comes as a single composition including at least:
i. an inhibitor of the HMG-CoA-reductase enzyme, and
ii. citrulline;
in a physiologically acceptable medium.

4. A combination product for use according to claim 1, characterized in that it comes as a kit including:
(a) a composition comprising at least an inhibitor of the HMG-CoA-reductase enzyme, in a physiologically acceptable medium; and
(b) a composition comprising at least citrulline in a physiologically acceptable medium;
said compositions (a) and (b) each being provided in a form which is suitable to a sequential, a separate and/or a simultaneous administration with one another over time.

5. A combination product for use according to claim 1, characterized in that the active substances (i) and (ii) or the compositions (a) and (b) are in a medium adapted to oral administration.

6. A combination product for use according to claim 1, said product being aimed at subjects suffering from glucose intolerance.

7. A combination product for use according to claim 1, characterized in that it contains as an active substance (ii) at least a substance selected from the group consisting of D,L-citrulline, L-citrulline, D,L-citrulline malate, L-citrulline malate, L-citrulline monoacetate, L-citrulline hydrochloride, L-citrulline methylester, L-citrulline ethylester, L-citrulline-n-hexylester, L-citrulline (benzoylmethyl)ester, alpha-N-benzoyl-L-citrulline methylester, B-Boc-citrulline, N1-2,4-dinitrophenyl-D,L-citrulline, or a plant extract of the cucurbitaceae family.

8. A combination product for use according to claim 1, characterized in that the HMG-CoA reductase inhibitor is selected from the group consisting of statins of natural or synthetic origin, monacolins, berberin, and policosanol.

9. A combination product for use according to claim 1, characterized in that the HMG-CoA reductase inhibitor comes as a red yeast rice extract, or monascus purpureus.

10. A combination product for use according to claim 1, for the treatment of stressed or anxious subjects, and/or subjects having a diet with a high fat or a high sugar content, and/or a sedentary lifestyle, and/or suffering from hormonal imbalances and/or from a genetic susceptibility to excess weight and/or obesity, or taking prescription drugs which can cause weight gain.

11. A combination product for use according to claim 1, characterized in that the active substance (i) is administered at a daily dosage of from 10 to 80 mg/day, when using a synthetic statin and the active substance (ii) is administered at a daily dosage of from 2 to 5 g/day.

12. A combination product comprising active substances consisting in:
(i) a HMG-CoA reductase inhibitor in the form of a plant extract containing the same, preferably a red yeast rice extract, and/or a plant extract including monacolins and/or berberin and/or policosanols;
(ii) citrulline or one of the bioequivalents thereof, preferably in the form of a plant extract containing the same; said substances (i) and (ii) being in a form adapted to a sequential, a separate and/or a simultaneous oral administration with one another over time.

13. A cosmetic treatment method for improving or slimming the figure and/or for stimulating the loss of excess weight and/or of cellulite and/or for limiting the localized accumulation thereof, comprising the administration of a combination product such as defined in claim 12, and the renewal of said administration until the expected cosmetic effect is obtained.

14. A cosmetic treatment method according to claim 13, characterized in that the active substance (i) is administered at a dose ranging from 100 to 600 mg one to three times a day and citrulline is administered at a daily dose ranging from 2 to 5 g/day.

15. A combination product for use according to claim 2, characterized in that it comes as a single composition including at least:
i. an inhibitor of the HMG-CoA-reductase enzyme, and
ii. citrulline;
in a physiologically acceptable medium.

16. A combination product for use according to claim 2, characterized in that it comes as a kit including:
(a) a composition comprising at least an inhibitor of the HMG-CoA-reductase enzyme, in a physiologically acceptable medium; and
(b) a composition comprising at least citrulline in a physiologically acceptable medium;
said compositions (a) and (b) each being provided in a form which is suitable to a sequential, a separate and/or a simultaneous administration with one another over time.
